(19) United States
(12) Patent Application Publication
(10) Pub. No.: US 2015/0032006 A1
(43) Pub. Date: Jan. 29, 2015

(54) MONITORING DEVICE

(71) Applicant: Leszek A. Szalek, Sunnyvale, CA (US)

(72) Inventor: Leszek A. Szalek, Sunnyvale, CA (US)

(21) Appl. No.: 14/341,878

(22) Filed: Jul. 28, 2014

Related U.S. Application Data
(60) Provisional application No. 61/859,239, filed on Jul. 28, 2013.

(51) Int. Cl. A61B 8/00

(52) U.S. Cl. CPC A61B 8/4455; A61B 8/4236; A61B 8/4281; A61B 8/4472; A61B 8/46
USPC 600/459

(57) ABSTRACT

A wearable device monitoring body conditions is disclosed. The device consists of a single or plurality transducers, particularly, ultrasonic transducers, and electronic circuitry which are packaged in a low profile patch. The electronics controls a transducer or transducers for signal transmission and receiving, data processing and wire or wireless interfacing to external devices. Laying out the electronics on a flexible printed circuit which mounted along with the transducers to a bendable material, easily adaptable to the curvature of a surface enables attaching the device to curved bodies, such as a human body, for purpose of health conditions monitoring. Low profile of the devices makes it possible to be worn a round-the-clock without causing much inconvenience for a patient. Depending on the application, the transducers can be configured as fixed or movable, providing appropriate monitoring results.

### CROSS-REFERENCE TO RELATED APPLICATIONS

This application is a continuation of U.S. patent application No. 61/859,239, filed Jul. 28, 2013.

### FIELD OF THE INVENTION

The present invention relates generally to data acquisition and signal processing, more particularly to a low profile, flexible monitoring device that can adapt to curved surfaces especially those of human bodies.

### BACKGROUND OF THE INVENTION

There are a number of devices and systems proposed for data collection, processing and imaging applied to examining human bodies. Most of them are very large, especially those using physical phenomena of X-rays, magnetic resonance, electrons and sound. The ones that utilize sound waves for data imaging, medical ultrasonography, are smallest in size among imaging diagnostic equipment, yet still too bulky to be attached to a human body for an extended period of time. Ultrasound probes are stiff, too big and heavy for a round-the-clock monitoring and diagnostics. They are designed as handheld devices for manual operation by medical personnel during patient examination.

In ultrasonography, for example, a single transducer transmits and receives a signal from one location. To generate a two dimensional (2D) image, the signal beam is swept by mechanically swinging or rotating a transducer. Or, an array of transducers can be used to obtain a 2D representation of the slice of the body part. In order to obtain a three dimensional (3D) image, a series of 2D adjacent images need to be acquired. It requires adding one more degree of freedom to the transducer array or to the entire probe either by sliding them or by rotating around a pivot point.

There are applications where larger area of an abdomen, a pelvis, a torso or limbs need to be monitored and changing over time process needs to be observed, e.g. variations in kidney, liver, bladder, heart or lungs volume, to diagnose a critical condition. And the process needs to be monitored a round-the-clock. Presently used in medical practice monitoring devices are not suitable for this type of application since they require permanent assistance of skilled personnel and holding a probe in one position over a time period of a few hours. Even, if the human assistance was eliminated by an automated system, still it is inconvenient for a patient to stay motionless for hours thus making current systems impractical for this type of applications.

There are some inventions proposed which eliminate a need for assistance of a third person performing the examination and address the problem of patient comfort during lengthy monitoring process. This more recent art, though, presents a number of shortcomings such as limited area of the body being monitored, an inconvenience for a patient to wear them, difficulty of securing a device at a fixed location for length of time, or being impractical to use for an ultrasound application, to list a few.

Examples of garment device, as disclosed in the U.S. Pat. No. 5,353,793 and No. 6,551,252, comprise a set of straps, a body harness, and are inconvenient to be worn during sleep and cumbersome to wear during daily activities, limiting its application to rather ambulatory environment.

In one of the most relevant prior art disclosures, for example, the US Patent No. US20120065479, a wearable patch device with an ultrasound transducer sensory array is presented. Even though the proposed ultrasound source sensor comprises a few, at least two but no more than eight integrated transducers, it still covers very limited area of the body. As disclosed, the transducers work more like a source of a single rather dispersed signals. In such configuration, there is a danger of missing an organ being the target of examination or observing the organ's edge, in both case providing misleading information. A few such devices can be attached to various body locations but it complicates the system by multiplying the interfaces and increases the overall system cost.

Another approach to monitoring human health condition with portable ultrasound monitoring system can be found in the US Patent No. US20110319760, where the disclosed monitoring system utilizes a band like supporting means housing an ultrasound transducer array. This invention presents several shortcomings. The supporting means requires to be fasten thus making it cumbersome to apply to the abdominal or chest part of a body for an extended period of time, making it inconvenient to be worn by a patient. Besides, as disclosed it may slip from its initial position making monitoring results questionable. Possible configuration of locating the system in pockets of a coat does not guarantee a stable, fixed transducer position due to movements of the coat, again making the monitoring process unreliable.

Other inventions, which expand limits of the monitored body area by providing 2D or 3D scans by using spatial transducer arrays or swinging a single transducer are either handheld devices or are too bulky to be implemented in a wearable patch. An ultrasound monitoring system providing spatial images can be found in the U.S. Pat. No. 6,099,474. This invention presents a device deflecting ultrasound beam from a stationary transducer. The beam deflecting mechanism is comprised of many mechanical components making it complicated and unsuitable for wearable application where device size, simplicity and energy consumption are critical factors. Similar shortcomings can be found in the U.S. Pat. No. 5,152,294, where mechanical complexity and component configuration make the invention impractical for a wearable device.

The present invention seeks to provide a device that overcomes the described limitations.

### SUMMARY OF THE INVENTION

The main object of the present invention is to provide a compact, pouch like monitoring device that can be attached to curved bodies for the purpose of acquiring and processing data. In particular, a device utilizing the ultrasonography (ultrasound) technology that can be applied in situations, when a human body needs to be monitored over lengthy period of time without causing much inconvenience to its user.

To achieve the above object, in accordance with the present invention, a low profile, flexible and durable device implementing a single or a plurality of transducers and electronics processing their signals is assembled on bendable material that easily adapts to the curvature of the surface to which it is attached.

The device comprises a single or plurality of transducers, or transceivers, and electronic circuitry that transmits, receives and processes signals from the transducers. The terms “transducer” or “transceiver” as used herein, generally refer to a device capable of transmitting and receiving a signal. The components are assembled on a flexible printed circuit that is attached to a flexible yet strong plastic sheet that can easily bend and accommodate to the curvature of a surface. The plastic sheet provides support for the flexible printed circuit and serves as a mechanical structure holding the transducers. The whole device is covered by a sealed flat pocket made of a flexible and durable fabric, filled inside with a soft padding material, which protects the device from being destroyed during the normal course of usage. The flat layout of the components encapsulated in the flat sack forms a sort of a pouch or a patch that can be attached to a body with adhesive or other means keeping it in place. The adhesive is distributed around the edge on the surface attaching to the body thus forming a pocket to which a gel used in ultrasound imaging can be applied.

The transceivers can be fixed or moving, performing a swinging motion. The fixed transceivers transmit and receive signal in the same location. Multiple fixed transceivers distributed around the pouch area provide information on a spatial condition of the monitored object. While utilizing moving transceivers, a single transceiver scans a certain sector of the object thus providing a set of signals that can be processed into a wedge-like 2D image.

In the proposed embodiments the thickness of the pouch is determined by the tallest component which typically is a transducer. Since transducers can be rather short, i.e. not exceeding a few millimeters in length, the pouch turns into a low profile device. As such, is does not present more inconvenience to the user than any other pouch or patch applied to the body, thus not disturbing the user's normal daily activities or sleep at night.

In order to maintain a low profile of the monitoring device while using moving transducers, solutions of small size actuators are proposed. A transducer is mounted on a single axis gimbal that enables a pendulum motion. A piece of smart metal, also known as muscle wire, with a combination of a spring can be used to pull and release a gimbal lever thus causing the transducer to swing. When electric current is applied to the muscle wire, the wire heats up and contracts, pulling the transducer in one direction, then when current is turned off the wire cools down and relaxes enabling the load spring to pull the transducer in the opposite direction. Repeating the cycle keeps the transducer swinging in a pendulum motion. A more costly alternative to the muscle-wire but allowing a higher frequency duty cycle, a piezo-motor is proposed. Those skilled in the art will know that other compact actuators are possible.

In another embodiment, a transducer is mounted in a 2-axis gimbal, enabling its motion simultaneously along the X and Y axes. Swinging the transducer along one axis, e.g. the X-axis provides a wedge-like image in a single plane while introducing a motion along the Y-axis provides a set of such images reflecting spatial information inside a body.

Conditions detected inside the monitored body can be interfaced to the user via audible or visual means embedded inside the device or can be transmitted through a wire or wirelessly to an external device for further processing and interfacing. A combination of both solutions is also possible.

### BRIEF DESCRIPTION OF THE DRAWINGS

FIG. 1a is a top view of the monitoring device.

FIG. 1b is a perspective view of the monitoring device.

FIG. 2 is a lateral cross-section through the middle of the monitoring device.

FIG. 3a is a lateral cross-section through the device in a state when attached to a curved surface.

FIG. 3b is a top view of the device attached to a human body.

FIG. 4a is a lateral cross-section through the device illustrating the major layers and components.

FIG. 4b is a top view of the device illustrating the major components layout on a flexible printed circuit attached to the flexible base.

FIG. 5a is a side view of a single axis scanning transducer actuated by a muscle wire.

FIG. 5b is a side view of a single axis scanning transducer actuated by a piezo-motor.

FIG. 6a-6d are respectively perspective, top, front and left views of a 2-axis scanning transducer assembly.

### DETAILED DESCRIPTION

The present invention will be described in detail based on the embodiments illustrated in the drawings. The embodiments of the present invention described below are not intended to be exhaustive or to limit the invention to the particular embodiments disclosed in the following detailed description. Rather, the embodiments are described so that others, particularly those skilled in the art may appreciate and understand the principles and practices of the invention.

To achieve this object, this invention provides a device implementing a plurality of transducers spread around certain area, particularly a single transducer, and electronics generating, receiving and processing signals, assembled on a flexible printed circuit. The layout of the transducers and electronic components on a flexible printed circuit makes possible a low profile device adjustable to curved surfaces. Setting multiple transducers at different locations allows data acquisition from those locations thus providing monitoring of a certain space. The transducers can be set still, each providing a single vector of data from one location or can be actuated performing pendulum movement, providing a series of data vectors that form a 2D image of a space sector.

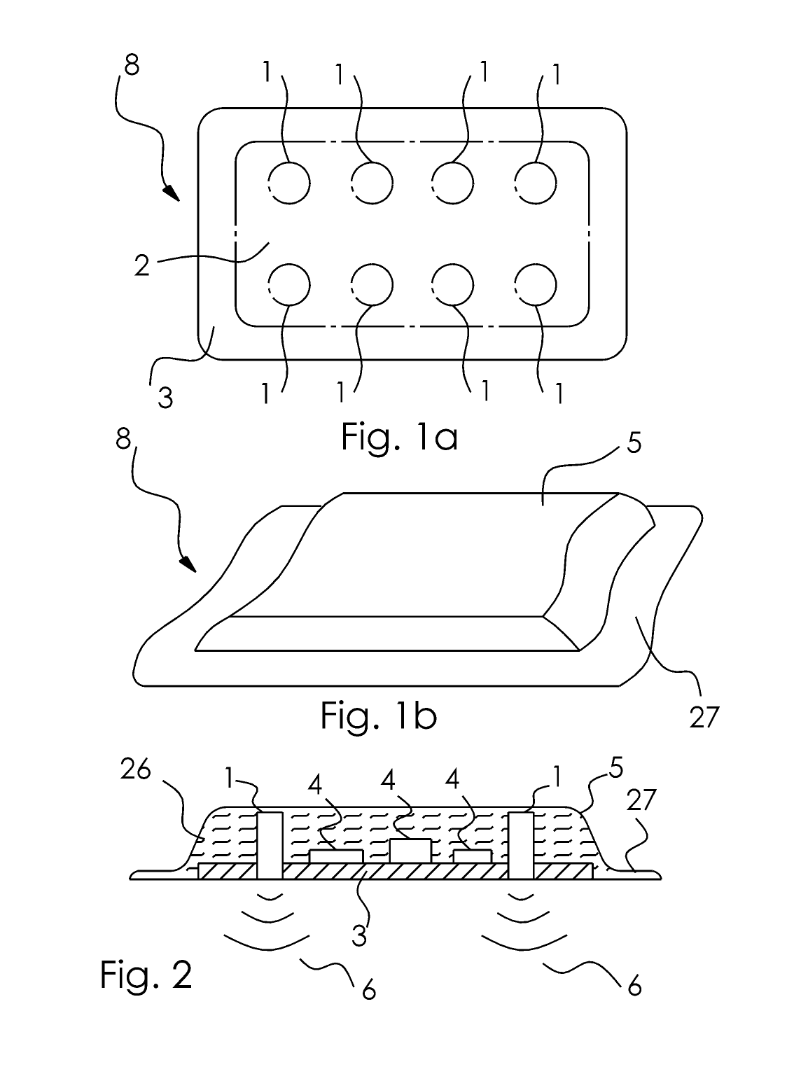

FIG. 1a is a top view of the monitoring device 8 capable of transmitting and receiving signals 6, as illustrated in FIG. 2, using plurality of transducers 1. The transducers are controlled by electronics assembled on a flexible printed circuit 2 which is attached to the surface of a flexible strong plastic sheet 3. The plastic sheet provides a stable structure for the electronic components which are covered by a waterproof durable fabric 5, as shown in FIG. 1b. The fabric is formed as a pocket and attached to the edges of the plastic sheet enclosing all the assemblies.

FIG. 1b is a perspective view of the monitoring device 8 showing its low profile and a patch like shape. It illustrates the flexible nature of the device allowing for applications that are not limited to only flat surfaces. The device 8 can be attached to the surface of a curved body using an adhesive substance distributed around the floppy edge 27 or by other means to keep the device in place.

FIG. 2 is a lateral cross-section through the middle of the device 8 showing the plastic sheet 3 with the mounted transducers 1 and electronic components 4 assembled on a flexible printed circuit. The sheet, made of flexible yet strong plastic, provides a stable support structure for the transducers transmitting and receiving signals 6. All the assemblies are protected and sealed inside a flat pocket 5. The floppy edges 27 of the pocket can be covered with an adhesive substance for the purpose of binding the pouch to external surfaces. The pocket is filled with soft padding material 26 for the purpose of protecting the internal components.

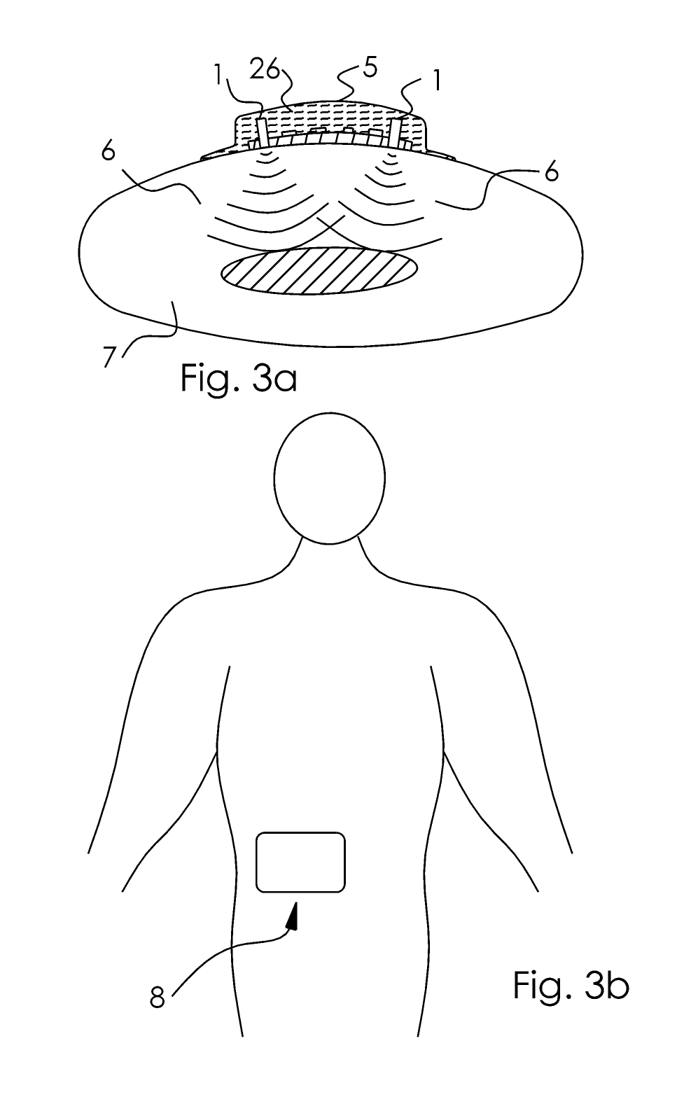

FIG. 3a is a side view of the monitoring device 8 attached to the curved surface of a body 7 which particularly can be a human body, as shown in FIG. 3b. The flexible nature of the support sheet 3 allows bending the device on curved surfaces without harm to its internal assemblies.

FIG. 3b is a top view of the monitoring device 8 attached to a human body. The device can be fixed by either an adhesive or some other means keeping it in place.

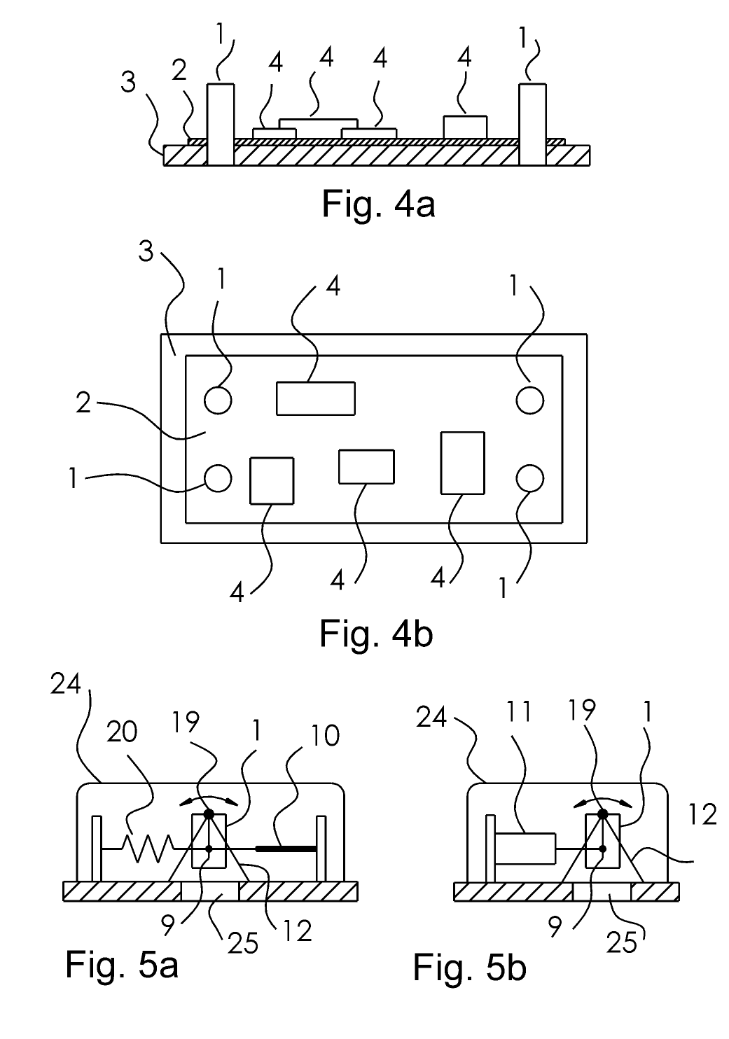

FIG. 4a is a side view of the transducer circuitry attached to the flexible base 3. The base provides a mechanical structure holding the transducers 1 and supporting the flexible printed circuit 2 on its surface. This way the circuitry is protected from braking and loosing the attached components 4. Enclosing the base 3 along with the flexible printed circuit 2 inside a durable cover protects the transducer system from being destroyed during the normal course of usage.

FIG. 4b is a top view of the transducers circuitry laid out on a flexible printed circuit 2. The flexible circuit is attached to the bendable plastic sheet 3 serving as the base structure providing a solid support for the transducers 1 and the flexible printed circuit 2 with the assembled electronic components 4.

FIG. 5a illustrates another approach the invention. It shows a side view of a single axis scanning transducer assembly. The transducer 1 is mounted to a gimbal 12 providing pivoted support for transducer pendulum movements around the axis 19. Such mounting allows scanning the space below the transducer within certain angular limits. The gimbal movement is actuated by a shape memory alloy element 10, so called muscle wire, which is counterbalanced by the spring 20. Both elements are attached to the gimbal lever 9 to generate pendulum movements of the transducer 1 when the muscle wire 10 contracts while heated by e.g. electric current flow and then relaxes, while cooling down, being pulled by the spring 20. The frequency of the pendulum movement is a function of the muscle wire heating and cooling process. The muscle wire is a very compact actuator, making possible for the gimbal structure to maintain its low profile, not exceeding height of the suspended transducer element. The moving elements assembly is covered by a hard cover 24 allowing for a free unobstructed motion inside and protecting the elements from damage.

FIG. 5b is a side view of a single axis scanning transducer assembly, showing possibility of using another compact actuator solution which in this case is a linear piezo-motor 11. The piezo-motor's rod pushes and pulls the gimbal lever 9 thus generating pendulum movements of the transducer 1. It is shown that applying a different type of the actuator, a low profile of the device 8 can be maintained. An opening 25 in the mounting base allows for signal transmission and receiving. It is understood that the opening needs to be covered by a protective screen enabling signal transmission.

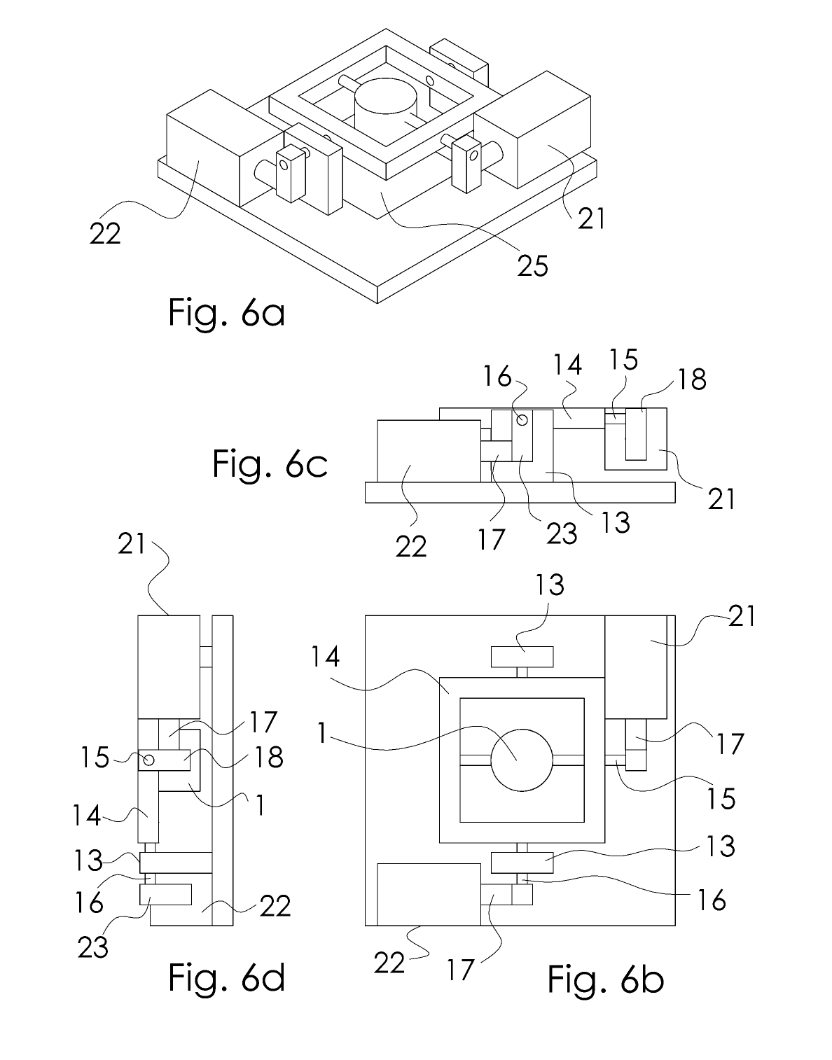

FIG. 6a illustrates another embodiment of the scanning device. The transducer 1 is mounted in a 2-axis gimbal mechanism. An actuator 21 pushes an arm mounted on one of the axis of the gimbal mechanism thus swinging the transducer along the X-axis while an actuator 22 pushes an arm attached to the other axis of the gimbal, swinging the transducer along the Y-axis. This way the transducer can be simultaneously swung along the perpendicular axes X and Y thus providing 3D images. The opening 25 in the mounting base allows for signal transmission and receiving.

FIG. 6b shows a top view of a 2-axis scanning transducer assembly. The transducer 1 is attached to a 2-axis gimbal 14 providing pivoted support for perpendicular pendulum movements of the transducer around the axes 15 and 16. The gimbal structure is supported by two support members 13, housing the pivot axis 16. The second pivot axis 15 is housed within the gimbal element 14, to which an actuator 21 is permanently attached. The second actuator 22 is permanently attached to the device base. This embodiment also requires a hard cover to allow for free unobstructed motion of the moving elements and to protect them from damage.

FIG. 6c is a front view of a 2-axis scanning transducer assembly exposing the actuator 22. The actuator's rod 17 pushes and pulls the gimbal lever 23 thus generating pendulum movement of the transducer 1 around pivot axis 16.

FIG. 6d is a left view of a 2-axis scanning transducer assembly exposing the actuator 21. The actuator's rod 17 pushes and pulls the gimbal lever 18 thus generating pendulum movement of the transducer 1 around the pivot axis 15.

As illustrated in FIG. 6a, FIG. 6b, FIG. 6c and FIG. 6d, two actuators can generate independent of each other perpendicular pendulum movements of the transducer element, thus enabling spatial angular scans of the object below. From a set of those scans a 3D image of the object can be integrated. In order to maintain a low profile of the gimbal structure, piezo-motors or other compact actuators should be used. Electronics accompanying the electro-mechanical scanning assembly can pre-process received signals and transmit them through a wire or wirelessly to an external device for further processing and image displaying.

The presented above solutions make the monitoring apparatus a practical device that can be applied to a human body for many hours minimizing an inconvenience for its user. The low profile and ability to adapt to curved surfaces become significant factors especially in case of an application to a human body, since they allow for easy concealment of the device under clothing and wearing it over extended period of time, a round-the-clock.

For certain applications a single or multiple still transducers are adequate. They transmit and receive a signal from the same location. The more transducers are used the higher resolution spatial information about the diagnosed object is provided. In some applications a scanning transducer providing 2D images can be desirable.

I claim:

1. A low profile flexible monitoring device, comprising a single or a plurality of fixed transducers, signal control and signal processing electronics assembled on a flexible printed circuit, a flexible support sheet, a durable fabric cover filled with a soft padding material.

2. A low profile flexible monitoring device as recited in claim 1, interfacing to the user via embedded audible or visual means.

3. A low profile flexible monitoring device as recited in claim 1, transmitting data through a wire or wirelessly to an external computing device for further processing and user interfacing.

4. A low profile flexible monitoring device, comprising a single moving transducer housed in a single axis gimbal, a compact actuator, signal control and signal processing electronics assembled on a flexible circuit, a flexible support sheet, a durable fabric cover filled with soft padding material.

5. A low profile flexible monitoring device as recited in claim 4, wherein said moving gimbal assembly is protected from damage by a sturdy cover.

6. A low profile flexible monitoring device as recited in claim 5, interfacing to the user via embedded audible or visual means.

7. A low profile flexible monitoring device as recited in claim 5, transmitting data through a wire or wirelessly to an external computing device for further processing and user interfacing.

8. A low profile flexible monitoring device, comprising a single moving transducer housed in a 2-axis gimbal, two compact actuators, signal control and signal processing electronics assembled on a flexible circuit, a flexible support sheet, a durable fabric cover filled with soft padding material.

9. A low profile flexible monitoring device as recited in claim 8, wherein said moving gimbal assembly is protected from damage by a sturdy cover.

10. A low profile flexible monitoring device as recited in claim 9, interfacing to the user via embedded audible or visual means.

11. A low profile flexible monitoring device as recited in claim 9, transmitting data through a wire or wirelessly to an external computing device for further processing and user interfacing.
